(19) United States
(12) Patent Application Publication
(10) Pub. No.: US 2008/0319510 A1
(43) Pub. Date: Dec. 25, 2008

(54) Medical Device Access Control Apparatus and Method

(76) Inventors: Fred A. Simpson, Houston, TX (US); John Tepper, Carrollton, TX (US)

Correspondence Address: FULBRIGHT & JAWORSKI, LLP, 1301 MCKINNEY, SUITE 5100, HOUSTON, TX, 77010-3095, US

(21) Appl. No.: 11/765,010

(22) Filed: Jun. 19, 2007

(51) Int. Cl. A61N 1/02

(52) U.S. Cl. 607/59

(57) ABSTRACT

A method and apparatus for providing access control to a medical device. A control device may be attached to the medical device to regulate power flow to the medical device and monitor usage of the device. The control device may accept removable media and read information from the media. The information may indicate control devices with which the removable media is designated to work, as well as an amount of authorization credit. The control device, after confirming that the removable media authorized to work with that control device and that it includes sufficient authorization credit, may provide power to the medical device. The control device may then monitor the medical device and when use of the medical device is detected, the control device may write new information to the removable media decreasing the amount of authorization credit. In a preferred embodiment, the removable media is an RFID memory card.

### TECHNICAL FIELD

The following relates generally to medical devices and more specifically to controlled access to a medical device.

### BACKGROUND OF THE INVENTION

Medical devices utilizing electromagnetic energy to diagnose or treat physiological symptoms have become common tools used by physicians and physical therapists in patient treatment. Such devices used for diagnostic purposes include x-ray machines, CT scanners, and MRI machines. Devices used for treatment purposes include Electromagnetic Field Therapy (EMFT) equipment used in treatment of soft tissue, Extracorporeal Shock Wave Lithotripsy (ESWL) equipment used to break up stones that form in the kidney, bladder, ureters, or gallbladder, Low-Level Laser Therapy (LLLT) equipment used to treat various conditions including Carpal Tunnel Syndrome, and the like.

An exemplary LLLT device is described in commonly-assigned U.S. Pat. No. 5,464,436 to Smith, which is hereby incorporated by reference in its entirety. A commercial embodiment of the technology described in U.S. Pat. No. 5,464,436 has been approved by the FDA for treatment of Carpal Tunnel Syndrome and is currently sold commercially as Microlight Corporation of America's “ML830®.”

Each of the above-mentioned devices are considered to be capital equipment often costing several thousands of dollars. Thus, to purchase a single piece of equipment, a physician or other practitioner must raise a large amount of capital before ever treating a patient with the equipment. This often proves difficult and limits the amount of equipment that a practitioner can obtain. Often, a practitioner will take out loans to purchase equipment. Other times, a practitioner will join a group where each member of the group pays for a small share of the equipment that is then shared among the group.

This capital equipment model also presents drawbacks to the medical device manufacturer. As discussed above, many practitioners are unable to purchase equipment because they lack the upfront capital, and thus the market for the devices is reduced. Additionally, while the manufacturer receives a large upfront sum for each piece of equipment sold, the manufacturer has no way to capture a continuing revenue stream. As a medical device saturates the market, this drawback becomes more acute as new sales decrease. Finally, while manufacturers or distributors have instituted programs where a medical device is leased for a number of years, such a program requires significant overhead to administer. For example, the manufacturer may be required to keep detailed records of all leased equipment, bill for payments, provide repair services, and recover equipment from delinquent lessees.

Turning to FIG. 1A, an exemplary prior art medical device 10 is shown. Medical device 10 is an LLLT device used for treatment of Carpal Tunnel Syndrome and is described more fully in U.S. Pat. No. 5,464,436. Medical device 10 includes a treatment portion 101, an annular switch 102, and a power portion 103. Treatment portion 101 includes a low-level laser in the embodiment of medical device 10, but could be any treatment apparatus used in medical capital equipment including, but not limited to, the exemplary electromagnetic capital equipment described above. Annular switch 102 is utilized to activate the device when pressure is applied to the switch. While an annular switch 102 is illustrated in conjunction with medical device 10, any appropriate user control may be utilized, such as a tactile switch. Power portion 103 is illustrated as a rechargeable battery in the embodiment of medical device 10, but could be any power source such as, for example, a standard plug designed to utilize standard AC power coupled by a wire to a voltage reduction device.

Medical device 10 is designed to implement a relatively simple treatment protocol and thus utilizes relatively simple internal circuitry and user controls. As shown in FIG. 1A, medical device 10 includes only a single user control, annular switch 102. When this switch is depressed, medical device 10 activates for a predetermined period of time—33 seconds in one embodiment. To implement such a simple treatment protocol, medical device 10 includes hard-wired control circuitry and need-not include more advanced, robust programmable control circuitry such as a microcontroller. This simple, hard-wired design presents difficulties in adding more advanced features to the device such as, for example, access control features, without completely redesigning the device. Such a complete redesign may be undesirable for several reasons including the costs and time to re-engineer, test, and fabricate a new product.

### BRIEF SUMMARY OF THE INVENTION

In one embodiment, the invention includes a control apparatus coupled to a medical device including a treatment portion. The apparatus may include a removable media reader configured to read information from a removable media and transmit the information to a processor and to receive information from the processor and write the information to the removable media, a power supply, and a power control unit coupled to the medical device and the power supply where the power control unit is configured to provide power to the medical device upon receiving an activation signal and is arranged such that the treatment portion of the medical device does not receive power except through the power control unit. The apparatus may also include a processor coupled to the removable media reader and the power control unit configured to receive information encoded on the removable media from the removable media reader, process the information, and provide an authorization signal to the power control unit, without contacting any device outside of the control apparatus and medical device, when the information indicates that the removable media is designated for use with the control apparatus and authorization credit remains for the removable media signal and is further configured to send information to the removable media reader responsive to a user using the treatment portion of the medical device where that information decreases the authorization credit recorded in the information on the removable media.

In another embodiment, the invention includes method for controlling access to a medical device having a power input and a treatment portion. The method may include the steps of connecting a control device to the medical device such that the power input of the medical device is connected to the control device and the power input is not accessible except through the control device, accepting at the control device a removable media, reading information from the removable media, and determining from the information whether the removable media has been designated to operate with the control device. The method may also include the steps of determining from the information, without contacting any device outside of the control apparatus and medical device, whether the removable media has any remaining authorization credit and if the removable media has remaining authorization credit, providing power, through the control device, to the medical device such that a user may use the treatment portion. The method may also include the steps of monitoring the medical device from the control device to determine if the treatment portion of the medical device is in use, and if it is determined that the treatment portion of the medical device has been used, writing information to the removable media where the information decreases the remaining authorization credit encoded on said removable media.

In another embodiment, the invention includes a method. The method may include the steps of coupling a control unit to the power input of a medical device, limiting user access to the medical device by providing power to the medical device from the control unit only when the control unit reads information authorizing use from a removable media, and monitoring use of the medical device from the control unit by monitoring, at the control unit, power provided to the medical device whereby if instantaneous power usage exceeds a threshold, the medical device is deemed to be in use.

In another embodiment, the invention includes a medical unit which may include a medical device and a control unit. The medical device may include a probe, an activation switch, and a driver circuit coupled to the probe and the activation switch configured to provide power to the probe responsive to the activation switch. The control unit may include a removable media reader configured to read information from a removable media and transmit the information to a processor and to receive information from the processor and write the information to the removable media, a power control unit coupled to the medical device and the power supply configured to provide power to the medical device upon receiving an activation signal arranged such that the treatment portion of the medical device does not receive power except through the power control unit, and a processor coupled to the removable media reader and the power control unit configured to receive information encoded on the removable media from the removable media reader, process the information, and provide an authorization signal to the power control unit when said information indicates that the removable media is designated for use with the control apparatus and authorization credit remains for the removable media. The processor may also be configured to provide the authorization signal without contacting any device outside of the control apparatus and medical device and to send information to the removable media reader responsive to a user using the treatment portion of the medical device where the information decreases the authorization credit recorded in the information on the removable media.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1A is an illustration of a prior art medical device;

FIG. 1B is a block diagram of the prior art medical device shown in FIG. 1A

FIG. 2 is a block diagram of a medical unit according to a preferred embodiment of the invention;

FIG. 3 is a block diagram of a medical unit according to a preferred embodiment of the invention;

FIG. 4 is an illustration of a physical implementation of the medical unit illustrated in FIGS. 2 and/or 3.

### DETAILED DESCRIPTION OF THE INVENTION

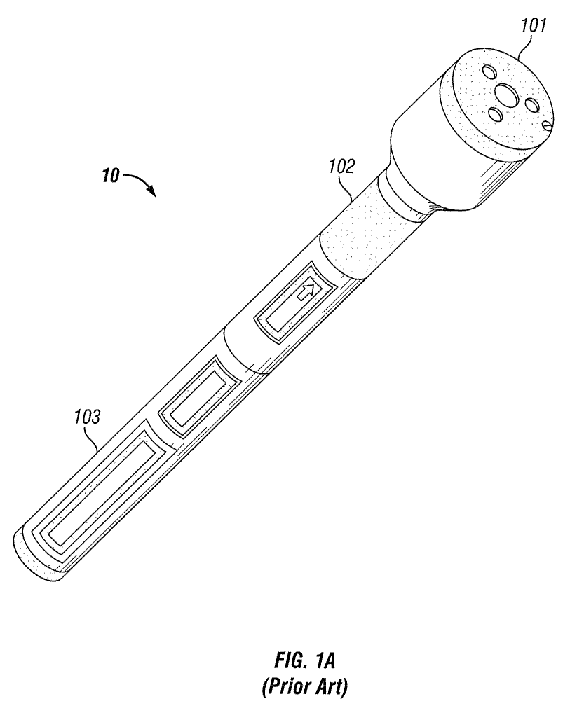

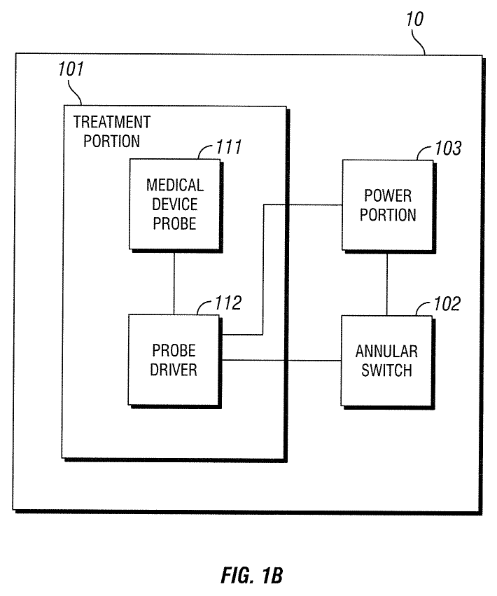

A block diagram of prior art medical device 10 is shown in FIG. 1B. While reference is made to medical device 10, medical device 10 is simply used in an exemplary manner. It will be understood by those skilled in the art that the methods and configurations discussed below may be adapted for use with various medical devices. Treatment portion 101 includes medical device probe 111 and probe driver 112. Medical device probe 111 consists of the actual electromagnetic excitation hardware. In medical device 10, the excitation may be provided by multiple GaAlAs laser diodes. Probe driver 112 includes circuitry to regulate the power supplied to medical device probe 111 thus regulating excitation of the probe. In medical device 10, this circuitry is relatively simple. The circuitry, responsive to a signal from annular switch 102, provides a set amount of power from power portion 103 to medical device probe 111 for a set period of time. This functionality may be implemented using basic circuitry without the need for any software-based processor such as a microprocessor.

According to one embodiment of the current invention, an existing medical device such as medical device 10, shown in FIG. 1B, is modified to restrict use of the medical device thus forming a controlled-access medical unit. A user may be required to utilize removable media such as an RFID memory card to operate the treatment portion of the medical device. The removable media includes a predetermined number of authorization credits. In a preferred embodiment, the removable media must stay within reading range of the medical unit while the unit is in operation. Once operation of the treatment portion is completed, new information may be written to the removable media reducing the number of authorization credits on the media based on the usage of the treatment portion.

By utilizing such a controlled-access medical unit that requires the use of removable media with credits that are decremented after use of the machine, a manufacturer can thus convert a medical device from typical capital equipment to a lower-priced device that provides the manufacturer a steady revenue stream through sales of removable media cards. In this arrangement, the equipment may be sold for little or no upfront compensation whereby compensation is later recovered through the sale of removable media having a predetermined number of credits. In this way, the manufacturer may charge for use of the machine and thus obtain a steady stream of later income. At the same time, the practitioner is not required to put forth any large capital outlays and instead only pays for actual use of the device. This actual use can be easily traced to and billed by the manufacturer to individual practitioner clients. Alternatively, the manufacturer may sell one or more of the removable media to the practitioner client and capture revenue immediately upon such sale. In one embodiment, a practitioner may utilize a different removable media for each patient, thus allowing easy tracking of use for billing and other purposes.

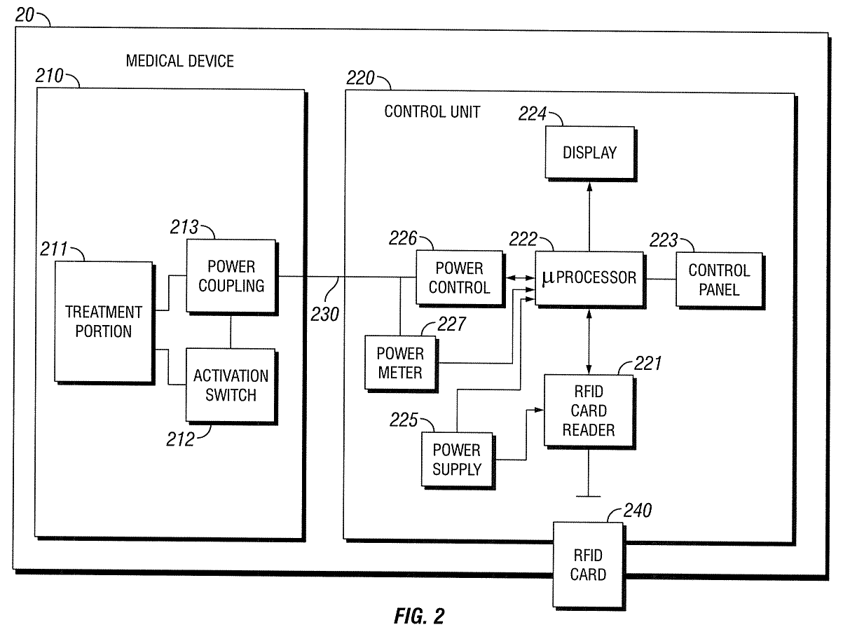

FIG. 2 illustrates a medical unit 20 according to a preferred embodiment of the invention. Medical unit 20 includes medical device 210 and a control unit 220. Medical device 210 in a preferred embodiment substantially comprises prior art medical device 10 modified as follows. The treatment portion 211 of medical device 210 remains unchanged from treatment portion 101. Similarly, activation switch 212 remains unchanged from annular switch 102. Thus, medical device 210 may include the same hard-wired treatment circuitry as was included in medical device 10. In place of power portion 103, power coupling 213 is provided. Power coupling 213 connects to control unit 220 over link 230. When power is provided to medical device 210 over link 230, medical device 210 operates in the same manner as prior art medical device 10. However, when power is not provided to medical device 210 over link 230, medical device 210, and in particular treatment portion 211, cannot operate. It should be understand that while this retrofitting procedure has been discussed with respect to prior art medical device 10, one skilled in the art would understand that this procedure may be easily adapted to other medical devices.

Control unit 220 provides power to medical device 210 over link 230 when authorized. Control unit 220 may include in a preferred embodiment an RFID card reader 221 capable of reading and writing to RFID memory card 240. While RFID technology is utilized in a preferred embodiment, it should be understood that any removable media technology may be utilized. RFID technology is desirable based, in part, on the wide-spread availability of RFID media and robust security features. Other common technologies, such as magnetic strip cards, may also be employed. In a preferred embodiment, the removable media provides information without the need to consult an external database such as, for example, over a phone line. Medical unit 20 may thus be securely operated without any need to contact any external source.

Control unit 220 may also include a microprocessor 222 or other similar processing unit to provide control, input/output, and analysis functions. These functions will be described in more detail below. RFID card reader 221 is coupled to microprocessor 222 through a digital communications line. Microprocessor 222 may also be coupled to a display 224 and a control panel 223. Display 224 and control panel 223, as will be understood by those skilled in the art, may include any number of standard components that carry out display and input functions, respectively. As will be discussed in detail below, the characteristics of display 224 and control panel 223 may be determined, at least in part, by the functions each component performs. In a preferred embodiment, user controls are limited such that control panel 223 consists of simply a power switch and display 224 is a basic display simply to display the remaining authorization credits (possibly converted to a more user-friendly form). In such an arrangement, control panel 223 may not be coupled to microprocessor 222, but may simply be arranged to regulate power supplied to components such as microprocessor 222 and RFID card reader 221.

Control unit 220 also includes, in a preferred embodiment, a power supply 225 coupled to RFID card reader 221, microprocessor 222, and power control circuitry 226. Power supply 225 may, in one embodiment, be a rechargeable battery similar to power portion 103 of medical device 10. Power supply 225 may also draw power from a standard AC power source or supply power in any other suitable way. Power supply 225 is capable of providing power to the components of control unit 220 as well as treatment portion 211 of medical device 210.

Power control circuitry 226 provides power from power supply 225 to medical device 210 over link 230 when signaled by microprocessor 222. Power control circuitry 226 may accomplish this function using any technique known to those skilled in the art such as, for example, utilizing a power transistor or, in a preferred embodiment, a relay. Additionally, control unit 220 may include power meter 227 for measuring the power provided to medical device 210. Power meter 227 may be integral with power control circuitry 226 or may be separate. In one embodiment, power meter 227 may include a precision resistor and an op-amp arranged to measure the current provide across link 230. This raw measurement may be passed on to microprocessor 222 for processing.

In a preferred embodiment, RFID memory card 240 may include two types of information. First, the card may include information detailing the number of authorization credits remaining on the card. Second, the card may include information about specific control units with which the card is authorized to work. For example, each medical unit manufactured may include a serial number. RFID memory card 240 may include a series of serial numbers corresponding to the serial numbers of the medical units owned by a specific client. The microprocessor may then read this list to determine if the card is authorized to work with that particular medical unit. In this way, a manufacturer may prevent cards sold to one practitioner from being used with another practitioner's device. Additionally, this check helps prevent counterfeiting and selling of the cards as a particular card will not work with the vast majority of medical units. Other categories of information may be included on RFID memory card 240, as well.

In a preferred embodiment, operation of medical unit 20 commences when a user selects an activation key on control panel 223. This causes microprocessor 222 to activate RFID card reader 221. When an RFID card such as RFID card 240 is placed in proximity to RFID card reader 221, RFID card reader 221 reads the information from the card and sends the information to microprocessor 222. Microprocessor 222 will read the information to determine if (a) the card is authorized to operate with medical unit 20, and (b) how many authorization credits remain. In a preferred embodiment, authorization credits represent units of time, such as minutes, of use of the treatment device. If the card is not authorized to operate with medical unit 20 or there are insufficient authorization credits remaining, microprocessor may instruct display 224 to display an error message. If the card is authorized to work with medical unit 20 and sufficient authorization credits remain, microprocessor 222 may send instructions to display 224 to display the remaining number of minutes corresponding to the available authorization credits on the card.

If microprocessor 222 determines that RFID card 240 is authorized to operate with medical unit 20 and sufficient authorization credits remain, microprocessor 222 signals power control circuitry 226 to provide power to medical device 210. When power control circuitry 226 provides power to medical device 210, treatment portion 211 is not automatically activated. Rather, medical device 210 is simply passively powered through power coupling 213. Treatment portion 211 may then be activated just as in the prior art version of the device such as, for example, by depressing activation switch 212. Power is continually provided to medical device 210 until RFID memory card 240 is removed or insufficient credits remain on the card.

In order to properly deduct authorization credit from the RFID card, control unit 220 must know when the treatment portion 211 is in use and when it is not. In a preferred embodiment, this determination is made by microprocessor 222 using measurements provided by power meter 227. As discussed above, power meter 227 measures the current provided to medical device 210 across link 230. This current will be significantly greater when treatment portion 211 is in use. In a preferred embodiment, power meter 227 represents the current provided by power control circuitry 226 as an analog voltage. This analog voltage is passed to an analog-to-digital converted and fed to microprocessor 222. The microprocessor may then compare this value to a threshold whereby if the value exceeds the threshold it is deemed that treatment portion 211 is in use. Microprocessor 222 may store in local memory the number of authorization credits read from RFID card 240 and may decrement this amount as it detects use. It may then write updated information to display 224 and RFID card 240.

While in a preferred embodiment control and monitoring of medical device 210 may be accomplished over a single link 230, in some embodiments, control unit 220 may utilize more advanced information available from medical device 210 or may replicate functions provided by medical device 310. For example, medical device 210 could include an “in use” indicator LED. Instead of using power meter 227, that indicator LED could be tapped to provide a signal to microprocessor 222 to indicate when treatment portion 211 is in use. Such a modification would complicate the system as it would require additional modifications to the existing medical device and would also complicate the connection between medical device 210 and control unit 220. However, such modification still allows treatment portion 211 to operate without being redesigned. As another example, an existing medical device may have its own display and control panel. These inputs may be removed from medical device 210 and may be “hosted” by display 224 and control panel 223. As yet another example, control unit 220 may perform value-added functions previously performed by external equipment or not available at all. In prior art medical device 10, the output power of the laser could only be measured by placing medical device 10 in a separate device that measured the output. In medical unit 20, the output power of the laser may be extrapolated from the power measurements of power meter 227 and may be displayed for the user on display 224.

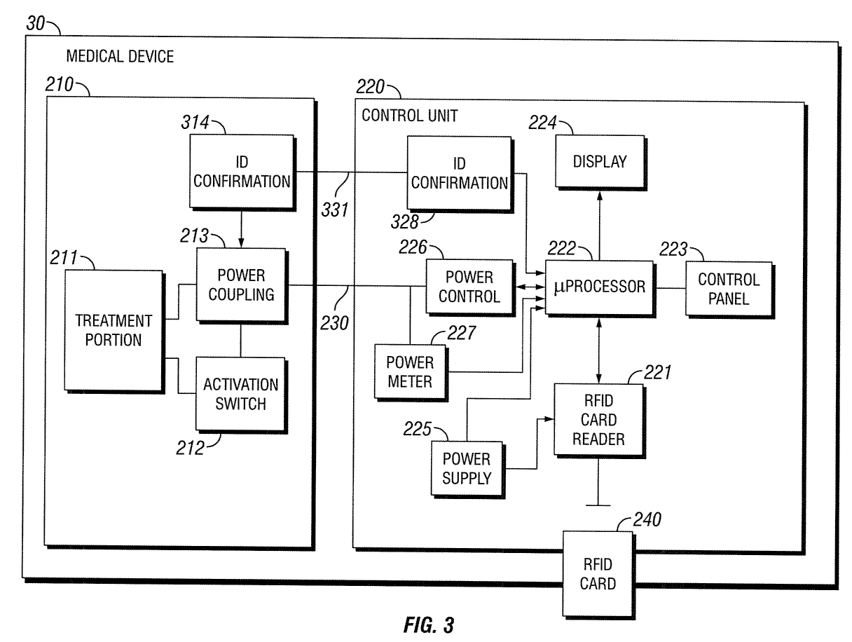

Turning to FIG. 3, another preferred embodiment of the invention is illustrated as medical unit 30. Medical unit 30 includes all of the components of medical unit 20 (and these components are labeled with like numbers) and also includes additional ID confirmation hardware. ID confirmation unit 314 in medical device 210 is coupled to ID confirmation unit 328 over link 331. Link 331 may be a separate communications link from link 230 utilizing, for example, a serial communications protocol or may optionally use the same physical link as link 230 and utilize a modulation technique to transmit data over link 230. ID confirmation unit 328 may be coupled to microprocessor 222 or may operate independently of microprocessor 222. ID confirmation unit 314 is coupled in a preferred embodiment to power coupling 213 to inhibit power from reaching treatment portion 211 even when power is provided over link 230 until ID confirmation unit 314 communicates with ID confirmation unit 328 and confirms that it is operating with an authorized control unit.

In one embodiment, ID confirmation unit 314 and ID confirmation unit 328 may be the same in all medical units produced. Such a system confirms that the medical device is being used in conjunction with some control unit and is not being directly powered to bypass the control features. In another embodiment, ID confirmation unit 314 and ID confirmation unit 328 may be customized such that medical device 210 will only operate with a specific control unit or class of control units. In another embodiment, ID confirmation unit 314, in addition to provide ID security, may provide information to control unit 220 about the make and model of medical device 210. Such a system would be helpful in allowing a single control unit to be used with several types of medical devices whereby the microprocessor properly determines what functions it must provide for a particular type of medical device based on the ID information received from ID confirmation unit 314. For example, power control circuitry 226 may need to provide different amounts of power to medical device 210 depending on the type of the device or microprocessor 222 may need to adjust the threshold it utilizes when analyzing power measurement data to determine if treatment portion 211 is in use.

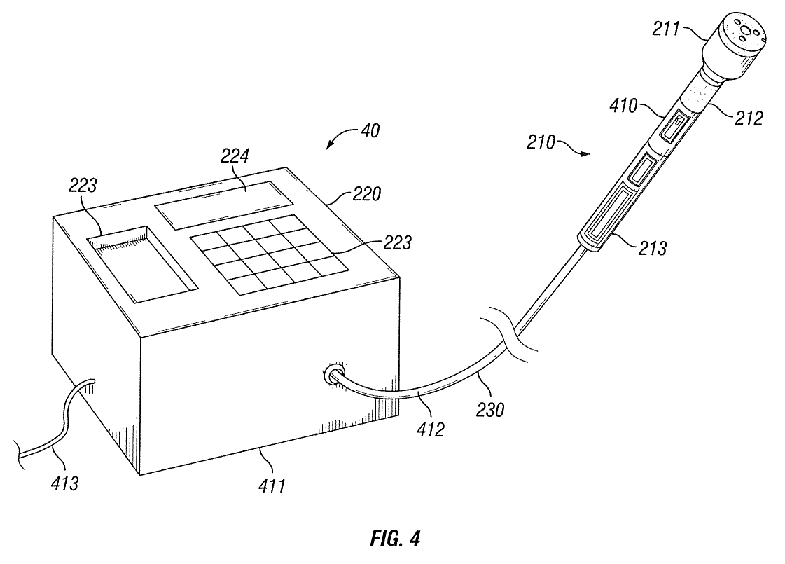

Turning to FIG. 4, a physical embodiment 40 of medical unit 20 or 30 according to a preferred embodiment is shown. Control unit 220 is realized in a console 411 while medical device 210 remains in shell 410. Link 230 is placed in insulated cable 412 extending from console 411 to shell 410. Shell 410 is the same shell used for medical device 10 modified to remove the power portion and to add power coupling 213 and an interface capable of accepting cable 412. This arrangement allows treatment portion 211 and actuator switch 212 to remain intact from prior art medical device 10. Console 411 includes RFID card reader 223, control panel 223, and display 224, as well as an output for cable 412. These components may be arranged in any manner as appropriate. Additionally, console 411 may include a power cable 413 capable of interfacing with a standard AC power outlet. Console 411 may utilize cable 413 as a constant power source or may simply utilize cable 413 to recharge an internal battery, thus allowing console 411 to operate without cable 413 being attached.

While physical embodiment 40 is shown as a two-piece unit connected by a cable, such an arrangement is merely exemplary. In other embodiments, it may be possible to integrate control unit 220 into an original shell of a medical device. In this case, microprocessor 222 may be relied upon to replicate input/output functions of the medical device using control panel 223 and display 224. Where control unit 220 is realized as a separate unit connected to the medical device by a cable, it may be preferable to include the ID confirmation circuitry included in medical device 30. Without such circuitry, a user may bypass control unit 220 by connecting cable 412 directly to a power source. Where control unit 220 is realized within the same shell as the portions of the medical device such that link 230 is not accessible to the user, it may be less necessary to include ID confirmation circuitry.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A control apparatus coupled to a medical device, said medical device comprising a treatment portion, said apparatus comprising:
a removable media reader configured to read information from a removable media and transmit said information to a processor and to receive information from said processor and write said information to said removable media;
a power supply;
a power control unit coupled to said medical device and said power supply; said power control unit configured to provide power to said medical device upon receiving an activation signal, said power control unit arranged such that said treatment portion of said medical device does not receive power except through said power control unit; and
a processor coupled to said removable media reader and said power control unit configured to receive information encoded on said removable media from said removable media reader, process said information, and provide an authorization signal to said power control unit when said information indicates that the removable media is designated for use with said control apparatus and authorization credit remains for said removable media wherein said processor is configured to provide said authorization signal without contacting any device outside of said control apparatus and medical device, said processor further configured to send information to said removable media reader responsive to a user using said treatment portion of said medical device, said information decreasing the authorization credit recorded in the information on said removable media.

2. The control apparatus of claim 1 wherein:
said removable media is encoded with information indicating identification information for control units with which the removable media is designated for use and information indicating authorization credit authorizing said processor to allow a user to operate said treatment portion of said medical device for a fixed period of time.

3. The control apparatus of claim 1 further comprising:
a power measurement device, said power measurement device configured to generate power measurement information indicating the power use of said medical device and to send said power measurement information to said processor;
wherein said processor is configured to interpret said power measurement information to determine whether said treatment portion of said medical device is in use.

4. The control apparatus of claim 3 further wherein:
said power measurement device is configured to generate power measurement information by measuring the current provided to said medical device through said power control unit.

5. The control apparatus of claim 1 comprising:
an ID confirmation unit;
wherein said ID confirmation unit is configured to provide information to an ID confirmation unit in said medical device to confirm said medical device is operating with an authorized control apparatus.

6. The control apparatus of claim 5 wherein:
said ID confirmation unit in said medical device includes information about the model of the medical device within which said ID confirmation unit is contained; and
said processor is configured to receive and utilize said information about the model of the medical device.

7. The control apparatus of claim 1, wherein:
said control apparatus is contained in a console separate from said medical device; and
said power control unit is coupled to said medical device by a cable extending from said console to said medical device.

8. A method for controlling access to a medical device, said medical device having a power input and a treatment portion, said method comprising the steps of:
connecting a control device to said medical device such that said power input of said medical device is connected to said control device and said power input is not accessible except through said control device;
accepting at said control device a removable media;
reading information from said removable media;
determining from said information whether said removable media has been designated to operate with said control device;
determining from said information whether said removable media has any remaining authorization credit, said determination made without contacting any device outside of said control apparatus and medical device;
if said removable media has remaining authorization credit, providing power, through said control device, to said medical device such that a user may use said treatment portion;
monitoring said medical device from said control device to determine if said treatment portion of said medical device is in use;
if it is determined that said treatment portion of said medical device has been used, writing information to said removable media, said information decreasing the remaining authorization credit encoded on said removable media.

9. The method of claim 8 wherein said step of monitoring said medical device from said control device to determine if said treatment portion of said medical device has been used comprises:
monitoring the current provided to said medical device by said control device such that if the current exceeds a threshold value, it is determined that the treatment portion of the medical device is in use.

10. The method of claim 8 comprising:
when said removable media is removed from said control device, inhibiting the use of said treatment portion of said medical device by cutting off power, at said control device, to said medical device.

11. The method of claim 8 comprising:
providing from said control apparatus a signal to said medical device, said signal indicating to said medical device that a control unit is connected to said medical device wherein said medical device is configured not to operate unless said control signal is received.

12. A method comprising:
coupling a control unit to the power input of a medical device;
limiting user access to said medical device by providing power to said medical device from said control unit only when said control unit reads information authorizing use from a removable media; and
monitoring use of said medical device from said control unit by monitoring, at said control unit, power provided to said medical device whereby if instantaneous power usage exceeds a threshold, said medical device is deemed to be in use.

13. The method of claim 12 comprising:
decreasing an authorization credit amount stored on said removable media based on the amount of time said medical device is deemed to be in use.

14. A medical unit comprising:
a medical device comprising:
a probe;
an activation switch; and
a driver circuit, coupled to said probe and said activation switch, said driver circuit configured to provide power to said probe responsive to said activation switch; and
a control unit comprising:
a removable media reader configured to read information from a removable media and transmit said information to a processor and to receive information from said processor and write said information to said removable media;
a power control unit coupled to said medical device and said power supply; said power control unit configured to provide power to said medical device upon receiving an activation signal, said power control unit arranged such that said treatment portion of said medical device does not receive power except through said power control unit; and
a processor coupled to said removable media reader and said power control unit configured to receive information encoded on said removable media from said removable media reader, process said information, and provide an authorization signal to said power control unit when said information indicates that the removable media is designated for use with said control apparatus and authorization credit remains for said removable media wherein said processor is configured to provide said authorization signal without contacting any device outside of said control apparatus and medical device, said processor further configured to send information to said removable media reader responsive to a user using said treatment portion of said medical device, said information decreasing the authorization credit recorded in the information on said removable media.

15. The medical unit of claim 14 wherein:
said removable media is encoded with information indicating identification information for control units with which the removable media is designated for use and information indicating authorization credit authorizing said processor to allow a user to operate said treatment portion of said medical device for a fixed period of time.

16. The medical unit of claim 14 wherein said control unit further comprises:
a power measurement device, said power measurement device configured to generate power measurement information indicating the power use of said medical device and to send said power measurement information to said processor;
wherein said processor is configured to interpret said power measurement information to determine whether said treatment portion of said medical device is in use.

17. The medical unit of claim 14 wherein said medical device further comprises:
an ID confirmation unit arranged to inhibit power from reaching said driver circuit until receiving ID confirmation information from said control unit.

18. The medical unit of claim 17 wherein said control unit further comprises:
an ID confirmation unit configured to provide confirmation information to said ID confirmation unit in said medical device.

19. The medical unit of claim 17 wherein:
said ID confirmation unit in said medical device includes information about the model of the medical device within which said authorization device is contained; and
said processor is configured to receive and utilize said information about the model of the medical device.

20. The medical unit of claim 14, wherein:
said control unit is contained in a console separate from said medical device; and
said power control unit is coupled to said medical device by a cable extending from said console to said medical device.
